(19) United States
(12) Patent Application Publication
(10) Pub. No.: US 2021/0299157 A1
(43) Pub. Date: Sep. 30, 2021

(54) CELLULOSE-BASED TOPICAL FORMULATIONS

(71) Applicant: Yissum Research Development Company of the Hebrew University of Jerusalem Ltd., Jerusalem (IL)

(72) Inventors: Oded SHOSEYOV, Shoham (IL); Tal BEN SHALOM, Beer Yaakov (IL)

(73) Assignee: Yissum Research Development Company of the Hebrew University of Jerusalem Ltd., Jerusalem (IL)

(21) Appl. No.: 17/266,177

(22) PCT Filed: Aug. 6, 2019
(86) PCT No.: PCT/IL2019/050896

§ 371 Date: Feb. 5, 2021

Related U.S. Application Data
(60) Provisional application No. 62/714,958, filed on Aug. 6, 2018.

(51) Int. Cl. A61K 31/717; A61K 9/00; A61K 8/73; A61K 8/02; A61P 17/04

(52) U.S. Cl. CPC A61K 31/717; A61K 9/0014; A61K 8/731; A61K 8/022; A61K 2800/75; A61K 2800/412; A61K 2800/413; A61K 2800/874; A61P 17/04

(57) ABSTRACT

The technology concerns topical formulations including nanocellulose materials for application on a skin or tissue of a subject.

### TECHNOLOGICAL FIELD

The technology subject of the invention disclosed herein concerns topical formulations comprising cellulose-based materials such as cellulose nanocrystals and oxidized forms thereof.

### BACKGROUND

Atopic dermatitis (AD), a common hereditary form of eczema, is a chronic, inflammatory skin disorder characterized by disruption of the epidermal barrier function and an aberrant immune response to antigens. It generally presents in infancy with most cases beginning before the age of five and tends to flare up periodically. It is characterized by severely itchy skin (pruritus) that results in redness and swelling. AD lesions may appear as fluid-filled vesicles that ooze, crack, and crust. Pruritus of the skin can cause frequent scratching and may result in lichenification (thickening of the skin) and secondary skin infections. It typically involves the popliteal (folded skin behind the knees) and the antecubital (in front of the elbows) areas, but can also affect the face, neck, and hands.

AD results in impaired barrier function and reduced water-holding capacity of the skin; this causes dry skin that requires treatment with specific bathing, cleansing, and moisturizing practices.

AD signs and symptoms vary widely from person to person and include:
Dry skin
Itching, which may be severe, especially at night
Red to brownish-gray patches, especially on the hands, feet, ankles, wrists, neck, upper chest, eyelids, inside the bend of the elbows and knees, and in infants, the face and scalp
Small, raised bumps, which may leak fluid and crust over when scratched
Thickened, cracked, scaly skin
Raw, sensitive, swollen skin from scratching.

There are several known deficiencies in the skin barrier of patients with AD. Two of the major risk factors that predispose a person to developing AD include a positive family history of atopy, a predisposition toward developing certain allergic hypersensitivity reactions, and loss-of-function mutations in the filaggrin gene. In addition, decreased intercellular lipids, altered ratios of ceramides, fatty acids, and cholesterol, as well as reduced expression of tight junction proteins which individually or collectively are likely contributing to the increased trans-epidermal water loss are characteristically observed in this disease.

AD is the most common chronic inflammatory skin disease, affecting approximately 20% of children and 3% of adults, and it imposes a significant financial and societal burden because of the direct medical costs and decreased productivity of individuals with AD. The majority of these children will outgrow the condition by adolescence. It is common for children with AD to also develop asthma and/or hay fever. This process is referred to as the “atopic march” and AD is often the first step in the sequential development of these other atopic conditions.

The clinical manifestations of AD vary with age, with infants showing AD on the extensor surfaces of extremities, face, neck, scalp, and trunk. Children are typically affected on the flexural surfaces of extremities, neck, wrists, and ankles, while adolescents and adults are generally affected on the flexural surfaces of extremities and the hands and feet. Patients often experience worsening itching symptoms throughout the night, and this may result in sleep loss, which can result in detrimental effects pertaining to school or work. Individuals with AD may also suffer from the social stigma of having a highly visible condition. Overall, these patient experiences describe a physically and mentally exhausting condition that can result in anxiety, depression, and decrease in quality of life.

Itching is the major symptom associated with impact on quality of life. For example, a US-based survey found 91% (n=304) of patients with eczema experienced itching on a daily basis, and 36% of patients identified decreasing the amount of itch to be their primary treatment goal. Furthermore, itch has been associated with mental distress and increased risk for suicidal ideation in those with AD. Of note, emotional stress has also been shown to increase itching, implying a bidirectional relationship.

Sleep disturbance is a frequent consequence of itching and is experienced by approximately two-thirds of patients with AD. Patients with sleep disturbance report difficulty initiating and maintaining sleep, which leads to daytime fatigue. Children with AD who experience sleep disturbances are associated with higher rates of developing attention-deficit/hyperactivity disorder, headaches, and short stature. Sleep disturbances experienced by adults with AD are associated with poor overall health perception.

Complications of atopic dermatitis (eczema) may include:
Asthma and hay fever. Eczema sometimes precedes these conditions. More than half of young children with atopic dermatitis develop asthma and hay fever by age 13.
Chronic itchy, scaly skin. A skin condition called neurodermatitis (lichen simplex chronicus) starts with a patch of itchy skin. When one scratches the area, it becomes even itchier. Eventually, one may scratch simply out of habit. This condition can cause the affected skin to become discolored, thick, and leathery.
Skin infections. Repeated scratching that breaks the skin can cause open sores and cracks. These increase the risk of infection from bacteria and viruses, including the herpes simplex virus, as well as fungal infections.
Irritant hand dermatitis. This especially affects people whose work requires that their hands are often wet and exposed to harsh soaps, detergents, and disinfectants.
Allergic contact dermatitis. This condition is common in people with atopic dermatitis.
Sleep problems. The itch-scratch cycle can cause poor sleep quality.

Atopic dermatitis is a complex, chronic inflammatory skin disorder with a profound symptom burden, and substantially affects patients' quality of life. The common, chronic, relapsing-remitting inflammatory disease can be challenging to treat. While there is no cure for AD, there are several therapeutic options available to patients to manage the condition. The majority of patients treat AD using general skin care methods, avoiding skin irritants, and applying topical anti-inflammatory therapy. However, these common methods often fail to improve AD, leading patients to use off-label systemic therapy (e g , immunosuppressant therapy) or other therapies such as phototherapy. The goals of AD management are to prevent flares (episode of worsening of symptoms typically requiring escalation of treatment) and to effectively manage flares when they occur by preventing AD progression, however this is easier said than done.

The burden of AD appears to be related mainly to the limited methods of treatment. Furthermore, according to the AD treatment guidelines, there is no standard of care, and treatment should be tailored to an individual's needs.

U.S. Pat. No. 9,095,603 [1] discloses a method of treating allergies by administration of oxidized cellulose.

U.S. Pat. No. 9,018,189 discloses uses of microfibrillated cellulose (MFC) as an anti-inflammatory agent for the treatment of skin inflammation.

### BACKGROUND ART

[1] U.S. Pat. No. 9,095,603
[2] U.S. Pat. No. 9,018,189

### GENERAL DESCRIPTION

The inventors of the technology disclosed herein have discovered that most allergens have a binding site for cellulose, called a Carbohydrate Binding Module (CBM). When the epidermal surface is coated with cellulose, it binds the allergen, thereby preventing it from penetrating the epidermis and causing a cascade inflammatory response. By applying a nanocellulose material, as defined herein, to a skin region to be exposed to the allergen, the competitive binding of the nanocellulose material to expansin-like proteins is exploited to block or reduce the immune response to the allergens.

Without wishing to be bound by theory, when a skin region becomes exposed to an allergen such as pollen or dust mites, the skin often prevents the allergen from penetrating the epidermis. If the allergen succeeds in penetrating the skin through the tight junctions of the epidermis, it may enter the dermis layer. In healthy individuals, a Th1 response begins, with the individual tolerating the irritant with no reaction. In sensitive individuals, however, the penetration can cause a cascade Th2 response followed by an IgE response. When the skin is exposed again, the CBM of the allergen binds the IgE antibody and an immune response, leading to an allergic reaction such as an inflammatory response and histamine activation is observed. Such an immune response leads to the development of Atopic Dermatitis,

Formulations of the invention have been found to act as barriers or protective layers to allergen-skin exposure. The nanocellulose material present in formulations of the invention acts to either bind the allergen or to prevent its skin penetration, thereby preventing it from causing the cascade inflammatory response. Unlike cellulose or oxides thereof which have been proposed for similar applications (see for example reference [1]), skin tight films of nanocellulose materials used herein have been found superior both in their ability to bind allergens under different pH values and also in their ability to provide the subject user with a more beneficial experience. Use of an oxidized cellulose, such as that disclosed in reference [1], was found to provide the user with a feeling of discomfort due to its sticky feeling, and was also found substantially insufficient as allergen blocker at pH values greater than 3.5. At lower pHs, the oxidized cellulose was found white, not transparent to light and therefore not appealing.

As such, formulations of the invention comprising the nanocelluloses disclosed herein have been found superior and more beneficial as compared to formulations comprising oxidized celluloses or MFC in at least one of the following:
They form transparent films on the skin;
They present a superior feeling and comfort to the user;
They are easily applied to the skin;
They are easily washed off the skin;
They are more effective in their crystalline forms as compared to their amorphous forms;
They provide a greater surface area as compared to their weight and therefore are superior;
MFC, being different from the nanocelluloses used in accordance with the invention, in comparison, does not provide transparent films and cannot be applied to the skin;
They are better effective in acting as physical barriers to allergens;
They better associate with allergens;
and others.

Thus, the invention generally concerns a topical formulation for application onto a skin region or a subject's tissue, the formulation comprising at least one nanocellulose and optionally at least one cosmetically or pharmaceutically acceptable carrier.

Also provided is a formulation comprising at least one nanocellulose, as defined herein, wherein the formulation is provided in a form suitable for application directly or indirectly onto a skin region or a tissue of a subject (human or non-human)

As detailed herein, formulations of the invention are intended for topical, non-systemic application onto a skin region or a tissue of a subject for the purpose of forming a barrier film against approaching allergens. As such, the skin region or tissue onto which the formulation is to be topically applied may be any region of the human or animal skin, including hair and nails. The formulation may be also applied topically to the eye by means known in the field of ophthalmology, e.g., eye drops, topically to various mucosal membranes and also topically to the inner skin regions or tissue of the ear(s), by means of ear drops. Formulations of the invention are non-toxic and thus may be applied to any region of the human or animal body. Formulations of the invention are not intended for systemic administration or non-topical administration and therefore are not intended for injection, inhalation, oral consumption and other means of systemic delivery.

Further provided is a film-forming formulation comprising at least one nanocellulose, as defined herein, wherein the formulation is provided in a form suitable for forming a film onto a skin region of a subject (human or non-human)

Also provided is a formulation for application on a skin or tissue of a subject, the formulation comprising at least one nanocellulose and optionally at least one cosmetically or pharmaceutically acceptable carrier, wherein the at least one nanocellulose consists essentially nanofibrilar cellulose (NFC), oxidized NFC, crystalline nanocellulose (CNC), oxidized CNC or combinations thereof.

Typically, formulations of the invention, once applied onto a skin region, form a transparent film that maintains the visual appearance of the skin and which is absent of discoloration. In other words, the film is substantially transparent in that it does not substantially alter the color of the skin onto which it is applied. In forming formulations of the invention and depending on the tool or means of delivery of the formulations, the formulation ingredients may be added to delivery media such as gels and emulsions without imposing discoloration or lose of transparency.

As used herein, the at least one “nanocellulose” is a cellulose-based material selected from nanofibrilar cellulose (NFC or cellulose nanofibrils, CNF), crystalline nanocellulose (CNC) (also known as nanocrystalline cellulose or cellulose whiskers), and oxidized forms thereof.

The term ‘nanocellulose’ does not encompass cellulose. The term also excludes oxidized forms of cellulose. In other words, both cellulose and oxidized forms thereof are excluded and do not form a part of any formulation of the invention. However, as the nanocelluloses may be prepared or derived from cellulose or oxidized cellulose or any cellulose form, minute contaminating amounts of cellulose or oxide forms thereof may be inadvertently present. Thus, formulations of the invention are regarded as consisting essentially of nanofibrilar cellulose (NFC), oxidized NFC, crystalline nanocellulose (CNC), oxidized CNC or combinations thereof; and further essentially free of cellulose or cellulose oxides. In other words, formulations of the invention intended to be free of such materials, for the aforementioned reasons, may comprise up to 1 wt % of cellulose and/or cellulose oxide.

Notwithstanding the above and without wishing to be bound by theory, any effect disclosed herein is derived from the presence of the at least one nanocellulose and not from the presence of any contaminating material, e.g., cellulose or cellulose oxide. In some embodiments, therefore, formulations of the invention comprise at least one cellulose material, the cellulose material consisting NFC, CNC, an oxide form of NFC, an oxide form of CNC or mixtures of any of the aforementioned.

In some embodiments, the formulation comprises NFC or an oxidized form thereof.

In some embodiments, the formulation comprises CNC. In some embodiments, the formulation comprises oxidized CNC.

Nanofibrilar cellulose, NFC, (also known as CNF, Cellulose Nano Fibers) is a cellulosic material composed of at least one primary fibril, containing crystalline and amorphous regions. NFC contains nanofibers with very high aspect ratios and both crystalline and amorphous regions. NFC is typically produced mechanically. In some embodiments, the NFC has an aspect ratio greater than 50. In some embodiments, the NFC length is typically between 0.1 and 5 μm and a diameter between 5 and 60 nm.

In some embodiments, the NFC is prepared from Micro Cellulose Crystals (MCC) typically by an acid treatment. When MCC is mechanical treated it may also be converted to CNF. The mechanical diminution of MCC, for example, in a microfluidizer generates NFC having properties identical to those measured for NFC produced by other methods. As compared to MCC, NFC particles have increased surface area, due to their reduced size, thereby demonstrating increased allergen-binding properties.

CNC is a fibrous material produced from cellulose. The CNC is typically a high-purity single crystal, characterized by having at least 50% crystallinity. In some embodiments, the CNC is monocrystalline. In some embodiments, the CNC, produced as particles (e.g., as a crystalline material) from cellulose of various origins, is selected to be at least about 100 nm in length. In some embodiments, the particles are at most about 1,000 μm in length. In some embodiments, the CNC particles are between about 100 nm and 1,000 μm in length, between about 100 nm and 900 μm in length, between about 100 nm and 600 μm in length, or between about 100 nm and 500 μm in length.

In some embodiments, the CNC particles are between about 100 nm and 1,000 nm in length, between about 100 nm and 900 nm in length, between about 100 nm and 800 nm in length, between about 100 nm and 600 nm in length, between about 100 nm and 500 nm in length, between about 100 nm and 400 nm in length, between about 100 nm and 300 nm in length, or between about 100 nm and 200 nm in length.

The thickness of the CNC material may vary between about 5 nm and 50 nm. The particles of CNC may be selected to have an aspect ratio (length-to-diameter ratio) of 10 and more. In some embodiments, the aspect ratio is between 60 and 100.

In some embodiments, the CNC is selected to be between about 100 nm and 400 nm in length and between about 5 nm and 30 nm in thickness.

CNC may be used as commercially available or may be prepared according to known methodologies such as the process described in WO 2012/014213 or its equivalent US application, herein incorporated by reference.

Where NFC is concerned, the NFC particles may have the same dimensions and aspect ratios as disclosed above for CNC and may therefore be selected from the above indicated dimensions and aspect ratios in an independently equivalent fashion.

Oxidized forms of CNC and NFC, as defined herein, result from the treatment of CNC or NFC, respectively, under oxidative conditions. The resulting oxidized CNC and an oxidized NFC are endowed with greater surface charge densities as compared to the unoxidized forms. As the degree of oxidation may be varied and may depend on the oxidant used, the time of exposure and the conditions of oxidation, various oxidation levels of CNC and NFC may be achieved.

In some embodiments, the oxidized CNC or NFC has at least a part or all of the exocyclic hydroxymethylene groups oxidized to carboxylic acid or carboxylate groups. The oxidized forms may be synthetic, semi-synthetic or commercially attained. They may be in their crystalline forms, amorphous forms or may be in a combination of both forms.

In some embodiments, the oxidized CNC is a product of oxidation of CNC with at least one oxidizing agent. The oxidizing agent may be any such used in organic synthesis. In some embodiments, the oxidant is TEMPO (herein TEMPO-mediated oxidized CNC). In some embodiments, the at least one nanocellulose, e.g., CNC or an oxidized form thereof, is provided in the form of particles, e.g., nanoparticles or microparticles or mixtures thereof.

In some embodiments, the particles are of an average diameter of between 0.01 and 100 microns. In some embodiments, the particles are nanoparticles having an average diameter of between 10 and 500 nm.

Formulations of the invention may be in a form of a solution or a suspension that comprises particles or flakes of the at least one nanocellulose. In cases where the formulation comprises particles of the at least one nanocellulose, the particles may be selected amongst microparticles and nanoparticles, as defined.

In some embodiments, formulations of the invention may comprise the at least one nanocellulose, as defined, in an amount ranging between 0.1 and 5 wt %. In some embodiments, the amount is between 0.1 and 4, between 0.1 and 3, between 0.1 and 2, between 0.1 and 1, between 0.2 and 4, between 0.3 and 4, between 0.4 and 4, between 0.5 and 4, between 0.6 and 4, between 0.7 and 4, between 0.8 and 4, between 0.9 and 4, between 1 and 4, between 0.1 and 0.9, between 0.1 and 0.8, between 0.1 and 0.7, between 0.1 and 0.6, between 0.1 and 0.5, between 0.2 and 0.9, between 0.3 and 0.9, between 0.4 and 0.9, between 0.5 and 0.9, between 0.3 and 0.6 or between 0.4 and 0.6.

The formulations may comprise a cosmetically or a pharmaceutically acceptable carrier, such as a vehicle, an adjuvant, an excipient, or a diluent. Such carriers are well known to those who are skilled in the art and are readily available to the public. It is preferred that the pharmaceutically acceptable carrier be one which is chemically inert to the nanocellulose or to any other component of the formulation and one which has no detrimental side effects or toxicity under the conditions of use.

The formulations may be in the form of a gel, an ointment, an emulsion, a thick cream, a liniment, a balsam, a lotion, a foam, a mask, a shampoo, tonic means, a cleaner, a spray, ear drops, eye drops, a conditioner, a hair spray, a roll-on, a powder including liquid powder, compact powder, cosmetic pencil, wet wipes/application cloth to be used on the skin or in any other traditional form used in the field of cosmetology or dermatology.

In some embodiments, the formulation is contained in an applicator. The applicator comprising a formulation of the invention may be any applicator which enables direct or indirect application of the formulation onto a skin region. The applicator may in the form of a roller-ball applicator (a roll-on), a dispensing device for a cream or an ointment, a spray, a disposable or non-disposable applicator cloth, tissue or membrane, or any other applicator which comprises the formulation and form which an amount of the formulation may be dispensed, delivered or administered to the skin region.

To achieve an effective film of several nanometers, in some embodiments, a formulation may be applied to the skin to provide a wet film of about 10 microns. The amount of the nanocellulose in the formulation may be, as disclosed herein between 0.1 and 1.5 wt %.

Notwithstanding the type of applicator used or the means by which an amount of the formulation is delivered to the skin region, the invention contemplates the provision of forming a thin film of the at least one nanocellulose material, as defined, on a skin region of a subject; the thin film being effective as a barrier to allergens. By protecting the skin region, the thin film prevents the binding of allergens to IgE epitopes or prevent their skin penetration, thereby preventing the development allergen-mediated diseases or disorders or minimizing the effect that may result from exposure to such allergens.

Thus, the invention further provides uses of at least one nanocellulose, as defined herein, for the preparation of topical formulations:
for preventing allergen-mediated diseases or disorders, e.g., in a subject prone to such a disease;
for reducing the severity of an allergen-mediated disease or disorder by blocking further exposure to such allergen causing diseases;
for interfering with allergen-IgE interactions;
for delaying the onset or lessening the severity of allergic reactions;
for reducing a subject's sensitivity to allergens;
for any combination of two or more of the above utilities/indications.

In some embodiments, the at least one nanocellulose is used in a method of:
preventing allergen-mediated diseases or disorders, e.g., in a subject prone to such a disease;
reducing the severity of an allergen-mediated disease or disorder by blocking further exposure to such allergen causing diseases;
interfering with allergen-IgE interactions;
delaying the onset or lessening the severity of allergic reactions;
reducing a subject's sensitivity to allergens; or
for any combination of two or more of the above utilities/indications.

Methods of the invention involve administering an amount of a formulation according to the invention onto a skin region of a subject and allowing said formulation to form a thin film of the at least one nanocellulose on the skin region. The skin region may be any portion of the subject's skin including hair and nails. In some embodiments, the skin to be applied with a formulation of the invention is a skin region exposed to the environment. In some embodiments, the skin region is or includes the face and neck. In some embodiments, the skin region is or includes the face, neck or hands.

In some embodiments, the skin region is not the complete subject's skin. In some embodiments, the skin region is the complete subject's skin.

Methods of the invention are directed at providing a beneficial effect or therapeutic benefit to a subject, either short-term and/or longer-term; the beneficial effect being achieved by preventing or ceasing interaction of allergens with the skin region. Thus, the term “prevention” or any lingual variation thereof, refers to an inhibition of at least one cause that may be onset of an allergic reaction, the cause being, inter alia, a contact between an allergen and a skin region or binding of an allergen to an IgE epitope. In subjects already suffering from an allergen-mediated disease or disorder, a provision of “treatment” may be characterized by an improvement in the subjects condition; a reduction in the severity, frequency, duration or progression of one or more adverse symptoms or complications associated with the disease or disorder; and/or an inhibition, reduction, elimination, prevention or reversal of one or more of the physiological, biochemical or cellular manifestations or characteristics of the disorder or disease, including complete prevention of the disease or disorder.

Generally speaking, subjects to be administered with a formulation of the invention are subjects who are likely to be exposed to an allergen or who are susceptible to having an allergic reaction. Subjects who are at risk of having an allergic reaction include subjects having a predisposition towards an allergic reaction, or infection or exposure to an agent that is associated with an allergy or allergic reaction due to a genetic or an environmental risk factor. Subjects who are predisposed can be identified by a personal or family history, through genetic screening, tests appropriate for detection of increased risk, or exhibiting relevant symptoms indicating predisposition or susceptibility. The allergic reaction may be any such symptom or condition associated with, for example, an existing allergic condition, a symptom or condition associated with or caused by an allergic condition, an acute allergic episode, a latent allergic condition, and seasonal or geographical tendencies.

The allergen capable of mediating a disease or disorder may therefore be any foreign agent that is capable of inducing, promoting, or stimulating allergy, i.e., the hypersensitive state induced by an exaggerated immune response to the allergen. The allergen may be plant/tree pollens or spores, animal dander, house dust mite, dust, lint, mite feces, fungal spores, and cockroaches.

In some embodiments, the allergen is a weed, plant or tree pollen optionally selected amongst pollen of dandelion, goldenrod, nettle, sage, clover, ragweed, mug wort, pellitory, nettles, dock, Bermuda couch grass, sweet vernal grass, red and blue grasses, Johnson grass pollen, ryegrass, timothy grass, orchard grass, tall fescue, meadow fescue and red fescue, alder, oak, ash, cypress, olive, maple, cedar, western red cedar, elm, birch, hickory, poplar, American sycamore, walnut, tobacco and cotton.

Animal allergens, such as pet allergens, e.g., dog and cat allergens, may include skin, hair, various parasites and fungi.

An allergen-mediated disease or disorder may be one which is directly associated with the coming into contact with the allergen or, alternatively, in cases where although initially not mediated by the allergen, an existing condition may deteriorate due to exposure (short or long term) to the allergen. In other words, the disease or disorder may be a reflection of a relationship that exists between an exposure to an allergen and the induction of a symptom, a condition, a disorder or a disease (for example a disease which is caused by binding of IgE to an allergen causing immediate Type I allergic reaction) or of the existence of a secondary effect of the exposure which exacerbates a condition, a disorder or a disease that may have been initially caused by another factor (for example a disease that is caused by another factor aggravated by the IgE-allergen interaction). Examples of such a disease or a disorder are the inflammatory, allergic and non-allergic diseases or disorders of the skin.

As known in the art, an inflammatory condition, disorder or disease refers to one or more physiological responses that characterize or constitute inflammation. An allergy or an allergic condition refers to a hypersensitivity to an allergen. Such conditions, disorders and diseases include but are not limited to hives (urticaria), eczema, angioedema, onchocercal dermatitis, dermatitis, atopic dermatitis, contact dermatitis and swelling.

In some embodiments, the disease is atopic dermatitis.

The invention thus further provides uses and methods as disclosed herein for preventing atopic dermatitis or for reducing skin-symptoms associated therewith.

The invention further provides uses and methods of preventing contact between an allergen causing atopic dermatitis and a skin region.

Current therapies for atopic dermatitis focus on symptom management by restoring epidermal barrier function with short-term emollients and reducing inflammation via steroids. However, there are no available clinical options for the prevention of atopic dermatitis. While a number of non-pharmacological topical therapies exist for treating the symptoms of atopic dermatitis, the most common therapy is the use of cosmetic moisturizers. The use of moisturizers is important to combat dry skin through hydration and the prevention of trans-epidermal water loss. Moisturizers are routinely used to provide some barrier protection for the skin from irritants or allergens and can act to soften the skin; reduce itching; and minimize cracking, fissuring, and lichenification. There are, however, several drawbacks to using moisturizers which formulations of the invention overcome. These include:
multiple applications throughout the day; and
moisturizers contain a combination of emollients, humectants, and occlusive agents. Some of these may provide the user with a less than positive experience. For example, humectants sting when applied to open skin and are not useful in children with atopic dermatitis. Occlusive agents (e.g., petrolatum, dimethicone, mineral oil) provide a layer of oil on the surface of the skin thereby limiting the skin's air permeability causing the skin to perspire and restart the atopic dermatitis cycle.

Formulations of the invention may be adapted for treatment or prophylactic regimens and may thus be tailored for short term or long term. The formulations or methods of the invention may employ a single administration of any one formulation or multiple administrations, wherein the formulation is administered alone or in combination with other therapeutics or treatments.

In some embodiments, any one formulation of the invention may further comprise at least one active or non-active agent or may be administrated in conjunction with at least one active agent. The at least one active agent may be selected from anti-allergy drugs, anti-histamine drugs, smooth muscle cell relaxing agents (e.g., linalool, magnesium sulfate), mast-cell stabilizers, anti-IgE drugs, analgesics, hormones, steroids, anti-inflammatory drugs, antibiotics, anti-viral drugs, anti-bacterial drugs, anti-fungal drugs, selective or non-selective potassium channel activators (broncho dilatators), muscarinic M3 receptor antagonists, M2 receptor agonists, opioid receptor agonists, H3-receptor agonists (inhibit acetylcholine release), phospholipase A2 inhibitors, 5-lipoxygenase inhibitors, 5-lipoxygenase activating protein (FLAP) inhibitors, leukotriens modifier drugs, leukotriens receptor antagonists, phosphodiesterase inhibitors, immunomodulating agents (e.g., cyclosporine), antibodies against adhesion molecules, antagonists of tachykinins, mucus secretion inhibitors, anti-oxidative agents and oxygen radical scavengers.

Non-limiting examples of actives are dexamethasone, triamcinolone acetonide, beclomethasone, dipropionate, flunisolide, fluticasone propionate, prednisone, methylprednisolone, mometasone furoate, chlorcyclizine, chlorpheniramine, triprolidine, diphenhydramine hydrochloride, fexofenadine hydrochloride, hydroxyzine hydrochloride, loratadine, promethazine hydrochloride, pyrilamine, omalizumab, albuterol, pirbuterol, epinephrine, racepinephrine, adrenaline, isoproterenol, salmeterol, metaproterenol, bitolterol, fenoterol, formoterol, isoetharine, procaterol, penicillin G, ampicillin, methicillin, oxacillin, amoxicillin, cefadroxil, ceforanid, cefotaxime, ceftriaxone, doxycycline, chlortetracycline, minocycline, tetracycline, amikacin, gentamycin, kanamycin, neomycin, streptomycin, netilmicin, paromomycin, tobramycin, azithromycin, clarithromycin, erythromycin, ciprofloxacin, lomefloxacin, norfloxacin, chloramphenicol, clindamycin, cycloserine, isoniazid, rifampin, vancomycin, aztreonam, clavulanic acid, imipenem, polymyxin, bacitracin, amphotericin, nystatin, nevirapine, delavirdine, efavirenz, saquinavir, ritonavir, indinavir, nelfinavir, amprenavir, zidovudine, stavudine, larnivudine, didanosine, zalcitabine, abacavir, acyclovir, penciclovir, valacyclovir, ganciclovir, 1-D-ribofuranosyl-1,2,4-triazole-3 carboxamide, 9,2-hydroxy-ethoxy methylguanine, adamantanamine, 5-iodo-2′-deoxyuridine, trifluorothymidine, interferon, adenine arabinoside, benzoic acid, undecylenic alkanolamide, cyclopiroxolamine, polyenes, imidazoles, allylamine, thicarbamates, amphotericin B, butylparaben, clindamycin, econaxole, amrolfine, butenafine, naftifine, terbinafine, ketoconazole, elubiol, econazole, econaxole, itraconazole, isoconazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, fluconazole, flutrimazole, nystatin, pimaricin, flucytosine, natamycin, tolnaftate, mafenide, dapsone, caspofungin, actofunicone, griseofulvin, potassium iodide, cyclopirox, cyclopirox olamine, haloprogin, ketoconazole, linalool, undecylenate, silver sulfadiazine, undecylenic acid, undecylenic alkanolamide and Carbol-Fuchsin.

Other drugs may be cytokines and chemokines, particularly anti-inflammatory cytokines such as IL-4 and IL-10.

The invention further provides a tool for applying a formulation of the invention to a skin region of a subject, the tool being selected from a gel, an ointment, an emulsion, a thick cream, a liniment, a balsam, a lotion, a foam, a mask, a shampoo, tonic means, ear drops, eye drops, a conditioner, a cleaner, a spray, a hair spray, a roll-on, a powder including liquid powder, compact powder, cosmetic pencil, wet wipes or an application cloth.

Further provided is a kit comprising an applicator according to the invention or a tool according to the invention, and instructions of use.

Thus, the invention provides A topical formulation for application on a skin or tissue of a subject, the formulation comprising at least one nanocellulose and optionally at least one cosmetically or pharmaceutically acceptable carrier, wherein the at least one nanocellulose is selected from nanofibrilar cellulose (NFC), oxidized NFC, crystalline nanocellulose (CNC) and oxidized CNC.

Also provided is a formulation comprising at least one nanocellulose, the formulation being provided in a form suitable for application directly or indirectly onto a skin region or a tissue of a subject, wherein the at least one nanocellulose is selected from nanofibrilar cellulose (NFC), oxidized NFC, crystalline nanocellulose (CNC) and oxidized CNC.

Further provided is a topical formulation for application on a skin or tissue of a subject, the formulation comprising at least one nanocellulose and optionally at least one cosmetically or pharmaceutically acceptable carrier, the at least one nanocellulose consisting essentially nanofibrilar cellulose (NFC), oxidized NFC, crystalline nanocellulose (CNC), oxidized CNC or combinations thereof.

In all aspects of the invention, formulations are for use in forming a film of the at least one nanocellulose on a skin region of a subject. The film is substantially transparent.

In all aspects, the formulation may be comprising NFC or an oxidized form thereof, or the formulation may be comprising CNC or an oxidized form thereof.

For all aspects, in the oxidized CNC or oxidized NFC at least a part or all exocyclic hydroxymethylene groups are oxidized to carboxylic acid or carboxylate groups.

For all aspects, the oxidized CNC is a product of oxidation of CNC with at least one oxidation agent such as TEMPO.

For all aspects, the formulation may be in a form of a solution or a suspension comprising particles or flakes of the at least one nanocellulose. For all aspects, the particles may be selected amongst microparticles and nanoparticles.

For all aspects, the amount of the at least one nanocellulose is between 0.1 and 5 wt %, or the amount is about 0.5 wt %.

For all aspects, the formulation is in the form of a gel, an ointment, an emulsion, a thick cream, a liniment, a balsam, a lotion, a foam, a mask, a shampoo, tonic means, a cleaner, a spray, a hair spray, ear drops, eye drops, a conditioner, a roll-on, a powder including liquid powder, compact powder, cosmetic pencil, wet wipes or an application cloth.

For all aspects, the formulation may be contained in an applicator, which may be a roller-ball applicator (a roll-on), a dispensing device for a cream or an ointment, a spray, a disposable or non-disposable applicator cloth, or a tissue or membrane.

For all aspects, the formulation may be for the preparation of a topical formulation for preventing allergen-mediated diseases or disorders.

For all aspects, the formulation may be for the preparation of a topical formulation for reducing the severity of an allergen-mediated disease or disorder by blocking further exposure to such allergen causing diseases.

For all aspects, the formulation may be for the preparation of a topical formulation for interfering with allergen-IgE interactions.

For all aspects, the formulation may be for the preparation of a topical formulation for delaying the onset or lessening the severity of an allergic reaction.

For all aspects, the formulation may be for the preparation of a topical formulation for reducing a subject's sensitivity to allergens.

For all aspects, the formulation may be for use in a method for:
preventing allergen-mediated diseases or disorders; or
reducing the severity of an allergen-mediated disease or disorder by blocking further exposure to such allergen causing diseases; or
interfering with allergen-IgE interactions; or
delaying the onset or lessening the severity of an allergic reaction; or
reducing a subject's sensitivity to allergens; or
any combination of the above methods.

Also provided is a method for preventing or ceasing interaction of an allergen with a subject's skin region, the method comprising applying to the subject's skin region a formulation according to any aspect of the invention.

For all aspects, the formulation may be applied by using a gel, an ointment, an emulsion, a thick cream, a liniment, a balsam, a lotion, a foam, a mask, a shampoo, tonic means, a cleaner, a spray, a hair spray, ear drops, eye drops, a conditioner, a roll-on, a powder including liquid powder, compact powder, cosmetic pencil, wet wipes or an application cloth comprising the formulation.

For all aspects, the formulation may be applied by using an applicator comprising the formulation.

For all aspects, the formulation may be for preventing or inhibiting a contact between an allergen and a skin region or binding of an allergen to an IgE epitope.

For all aspects, the formulation may be applied onto a subject's skin region prior to exposure to an allergen or to a subject susceptible to having an allergic reaction.

For all aspects, the allergen may be capable of inducing, promoting, or stimulating allergy. For all aspects, the allergen may be a plant/tree pollen, animal dander, house dust mite, dust, lint, mite feces, fungal spores, and cockroaches. For all aspects, the allergen may be a weed, plant or tree pollen optionally selected amongst pollen of dandelion, goldenrod, nettle, sage, clover, ragweed, mug wort, pellitory, nettles, dock, Bermuda couch grass, sweet vernal grass, red and blue grasses, Johnson grass pollen, ryegrass, timothy grass, orchard grass, tall fescue, meadow fescue and red fescue, alder, oak, ash, cypress, olive, maple, cedar, western red cedar, elm, birch, hickory, poplar, American sycamore, walnut, tobacco and cotton.

For all aspects, the method may be for preventing an allergen-mediated disease or disorder or form arresting deterioration of an existing condition due to continued exposure to the allergen.

For all aspects, the disease or disorder is caused by binding of IgE to an allergen causing immediate Type I allergic reaction.

For all aspects, the disease or disorder is selected amongst inflammatory, allergic and non-allergic diseases or disorders of the skin.

For all aspects, the disease or disorder is selected from hives (urticaria), eczema, angioedema, onchocercal dermatitis, dermatitis, atopic dermatitis, contact dermatitis and swelling.

For all aspects, the disease or disorder is atopic dermatitis.

Also provided is a method for preventing atopic dermatitis in a subject, the method comprising applying to a skin region of the subject an amount of a formulation according to any aspect of the invention.

Also provided is a method for preventing contact between an allergen causing atopic dermatitis and a skin region of a subject, the method comprising applying to a skin region of the subject an amount of a formulation according to any aspect of the invention.

The invention further provides uses and methods as disclosed herein for preventing atopic dermatitis or for reducing skin-symptoms associated therewith.

Also provided is an applicator comprising a formulation according to any aspect of the invention.

Also provide is a tool for applying a formulation according to any aspect of the invention to a skin region of a subject, the tool being selected from a gel, an ointment, an emulsion, a thick cream, a liniment, a balsam, a lotion, a foam, a mask, a shampoo, tonic means, a cleaner, a spray, a hair spray, a roll-on, a powder including liquid powder, compact powder, cosmetic pencil, wet wipes or an application cloth.

Also provided is a kit comprising an applicator according to the invention or a tool according to the invention, and instructions of use.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. SDS-PAGE analysis of Resilin-CBD/CNC binding assay at pH 8. Resilin-CBD/CNC at weight ratios of 10:1. Control: pure Resilin-CBD T: Total resilin-CBD/CNC prior CENTRICON separation R: retentate post CENTRICON separation P: peremeate post CENTRICON separation.

FIG. 2. SDS-PAGE analysis of Resilin-CBD/CNC binding assay at pH 5.5. Resilin-CBD/CNC at weight ratios of 10:1. T: Total resilin-CBD/CNC prior CENTRICON separation R: retentate post CENTRICON separation P: peremeate post CENTRICON separation.

FIG. 3. SDS-PAGE analysis of Ambrosia soluble pollen proteins /CNC binding assay at pH 7.5. in v:v ratio of 1:3 and 1:10 for Ambrosia soluble pollen proteins: OXCNC and 1:3 v:v ratio for Ambrosia soluble pollen proteins: CNC T: Total Ambrosia soluble pollen proteins/CNC prior CENTRICON separation. R: retentate post CENTRICON separation. P: peremeate post CENTRICON separation. Control: pure Ambrosia soluble pollen proteins.

FIGS. 4A-C. Binding assays showing Lolium perenne pollen binding to different forms of cellulose, including oxidized cellulose and microcrystalline cellulose (FIG. A), oxidized cellulose (FIG. B) and microfluidized CNC (FIG. C).

### DETAILED DESCRIPTION OF EMBODIMENTS

### Carbohydrate Binding Modules (CBMs)

One of the mechanisms that nature evolved is Carbohydrate Binding Modules (CBMs) which bind structural proteins to polysaccharide scaffolds such as cellulose in the plant kingdom and chitin in the animal kingdom respectively.

With respect to cellulose, during the course of evolution, many microorganisms developed an array of carbohydrate degrading enzymatic complexes termed “Cellulosomes”. Apart from the hydrolytic units, these complexes usually contain Carbohydrate Binding Modules (CBMs) that anchor the hydrolytic enzymatic complexes to the cellulose, many allergens, such as grass and dust mite, contain a common CBM motif.

### Production of Crystalline Nano Cellulose

Currently, CNCs are mainly produced by acid hydrolysis/heat controlled techniques, with sulfuric acid being the most utilized acid. Extraction of the crystals from cellulose fibers involves selective hydrolysis of amorphous cellulose regions, resulting in highly crystalline particles with source dependent dimensions, e.g., 5-20 nm×100-500 nm for plant source CNCs. Sulfuric acid hydrolysis grafts negatively charged sulfate half-ester groups onto the surface of the particles, which act to prevent aggregation in aqueous suspensions due to electrostatic repulsion between particles. Furthermore, the rod-like shape of CNCs leads to concentration-dependent liquid crystalline self-assembly behavior.

### TEMPO-Mediated Oxidation of CNCs

Total of 45 gr (4 mmol of glucosyl units) of cellulose nanocrystals were suspended in water (3.46 L) containing 692 mg of 2,2,6,6-tetramethyl-1-piperidinyloxyradical (TEMPO, 0.065 mmol) and 13.84 gr of sodium bromide (1.9 mmol) at room temperature for 30 min The TEMPO-mediated oxidation of the cellulose nanocrystals was initiated by slowly adding 734.37 mL of 6% NaClO (8.6 mmol) over 20 min at room temperature under gentle agitation. The reaction pH was monitored using a pH meter and maintained at 10 by incrementally adding 0.5 M NaOH. When no more decrease in pH was observed, the reaction was considered complete. About 346 mL of methanol was then added to react and quench with the extra oxidant. After adjusting the pH to 7 by adding 0.5 M HCl, the TEMPO-oxidized product was washed with D.I. water by centrifugation and further purified by dialysis against D.I. water for two days.

### Carbohydrate-Binding Module Binding to OXCNC

Recombinant Resilin protein fused to cellulose binding domains was used to simulate the binding of CBM-containing allergens to OXCNC.

Binding was accomplished by incubating mixtures of res-CBD and CNCs at pH8 and pH 5.5 for 1 hours at room temperature, with gentle rotation of the sample. Mass ratios of 1:10 of res-CBD: CNCs were tested. After incubation, ultrafiltration was used to isolate unbound protein (EMD Millipore Centricon membranes, 0.2 μm pore size), since unbound res-CBD (MW≈53 KDa) passes through the membrane, whereas CNCs do not. The different mass ratio mixtures were centrifuged using a bench-top model (14000 rpm), and the permeates and retentates were collected and analyzed by SDS-PAGE to determine conditions for optimal binding. In addition, pure res-CBD was used as a control in order to establish non-specific interactions between the proteins and the membrane, while pure CNC was used in order to establish that CNC does not pass through the membrane. Equal amounts of protein (3.5 μg) from the total mixture (T), retentate (R), and permeate (P) of each of the tested mass ratios were loaded and separated on the acrylamide gel (12.5%), and compared to a protein ladder (7 μL, MW range 250-10 KDa) and to a sample of pure res-CBD (3.5 μg).

### Resilin-CBD/CNC Binding Assay

Resilin-CBD/OXCNC in different weight ratios were incubated followed by protein separation via CENTRICON membrane with a CO of 0.22 μm. Permeats (˜100 μL) and Retentate (˜100 μL) were collected and analyzed by SDS-PAGE (12.5%). 1:10 wt. ratio of resilin-CBD/CNC is˜the maximum binding capacity using these conditions. Previous gravimetric tests indicated that unlike resilin-CBD, CNCs do not permeate the membrane to any measureable extent.

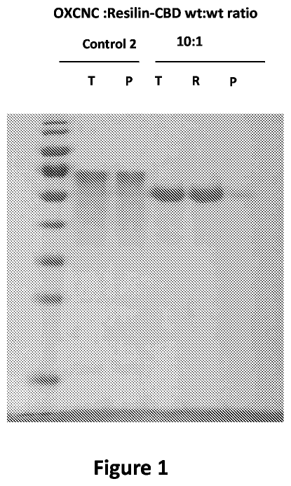

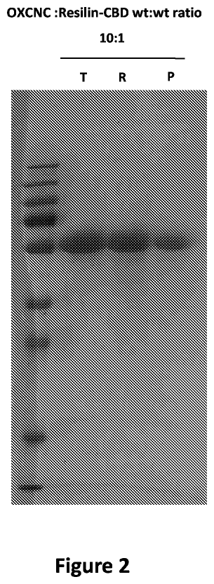

It was found by SDS-PAGE analysis that the optimal binding of res-CBD to OXCNCs under the conditions used in this work was pH8 (FIG. 1), at Ph5.5 about 50% of the protein binds to OXCNC (FIG. 2) while in pH 8, about 90% of the protein binds to OXCNC at weight ratios of 10:1. At lower weight ratios, we will probably obtain 100% protein binding to OXCNC.

### Pollen Allergenic Proteins

Ambrosia pollen is notorious for causing allergic reactions in humans, specifically allergic rhinitis. Up to half of all cases of pollen-related allergic rhinitis in North America are caused by Ambrosia. (Taramarcaz, P.; et al. (2005). “Ragweed (Ambrosia) progression and its health risks: will Switzerland resist this invasion?” Swiss Medical Weekly. 135 (37/38): 538-48.)

### Pollen Proteins Extraction

Native Ambrosia (A. confertiflora) pollen from wild plants suspended in PBS×1 buffer and incubated at room temperature for 20 min, and then centrifuged. The supernatants was separated from the pellet and used for allergen-binding assay to OXCNC and CNC.

### Allergen-Binding Assay

Binding was accomplished by incubating mixtures of different mass ratios of soluble pollen proteins and OX CNC or CNC at pH7.5 for 1 hours at room temperature, with gentle rotation of the sample. Volumetric ratios of 1:3 and 1:10 of soluble pollen proteins: CNCs were tested. After incubation, ultrafiltration was used to isolate unbound protein (EMD Millipore Centricon membranes, 0.2 μm pore size), since unbound soluble pollen proteins passes through the membrane, whereas CNCs do not. The different Volumetric ratio mixtures were centrifuged using a bench-top model (10000 rpm), and the permeates and retentates were collected and analyzed by SDS-PAGE to determine conditions for optimal binding.

### Binding of Pollen Allergenic Proteins to O×CNC and CNC

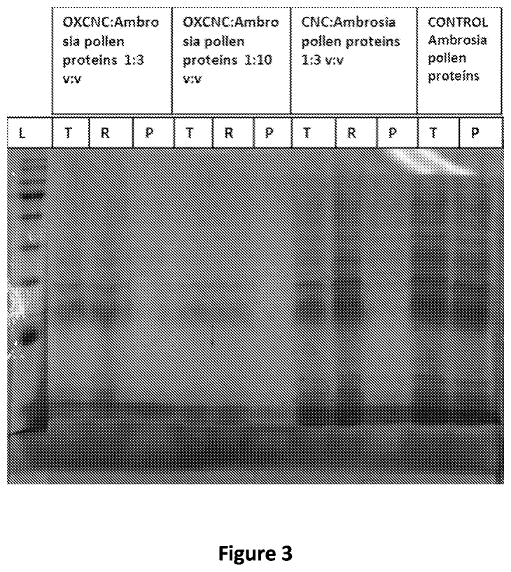

As FIG. 3 demonstrates, Ambrosia soluble pollen proteins passes easily through the membrane (CONTROL Ambrosia pollen proteins). Previous gravimetric tests indicated that unlike the soluble pollen proteins, CNCs or OXCNCs do not permeate the membrane to any measureable extent. Soluble pollen proteins that were incubated with OX CNC or CNC at pH7.5 bind to the CNC partial and remain with the OXCNC or CNC particles at the retentate in v:v ratio of 1:3 and 1:10.

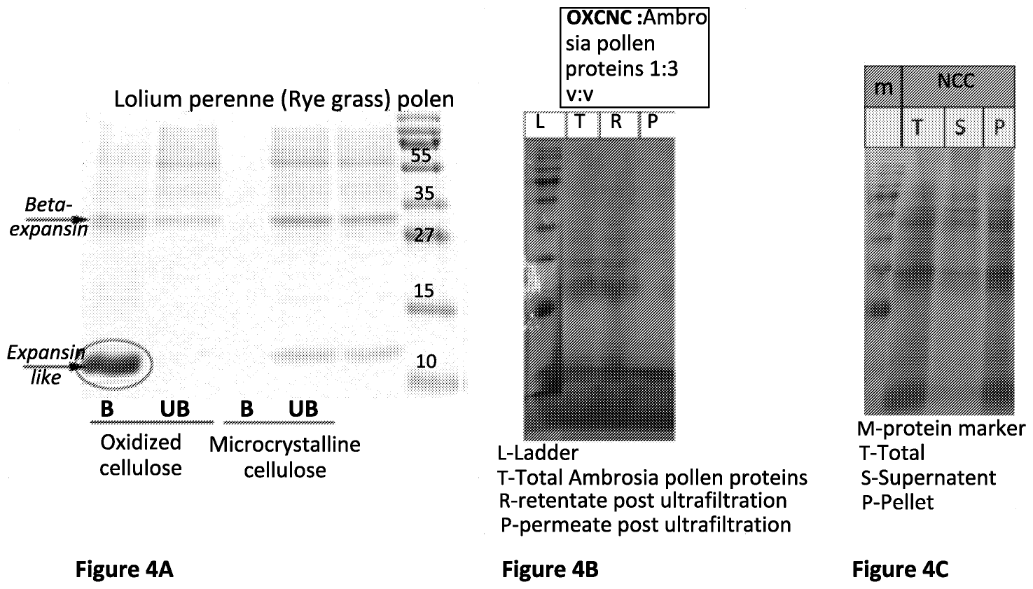

Following the procedure in the microfluidizer, binding assays were performed using the same pollen as used to check the oxidized cellulose. Results show that the proteins in the pollen did indeed bind to the oxidized cellulose. Binding assays showing Lolium perenne pollen binding to different forms of cellulose, including oxidized cellulose and microcrystalline cellulose, oxidized cellulose and microfluidized CNC are shown in FIGS. 4A-C.

1.-40. (canceled)

41. A topical formulation comprising at least one nanocellulose and optionally at least one cosmetically or pharmaceutically acceptable carrier, wherein the at least one nanocellulose is selected from nanofibrilar cellulose (NFC), oxidized NFC, crystalline nanocellulose (CNC) and oxidized CNC.

42. The formulation according to claim 41, provided as a film of the at least one nanocellulose on a skin region of a subject.

43. The formulation according to claim 41, the formulation comprising CNC or an oxidized form thereof.

44. The formulation according to claim 41, wherein the oxidized CNC is a product of oxidation of CNC with TEMPO.

45. The formulation according to claim 41, the formulation being in a form of a solution or a suspension comprising particles or flakes of the at least one nanocellulose.

46. The formulation according to claim 41, the formulation being in the form of a gel, an ointment, an emulsion, a thick cream, a liniment, a balsam, a lotion, a foam, a mask, a shampoo, tonic means, a cleaner, a spray, a hair spray, ear drops, eye drops, a conditioner, a roll-on, a powder including liquid powder, compact powder, cosmetic pencil, wet wipes or an application cloth.

47. The formulation according to claim 41, being contained in an applicator.

48. The formulation according to claim 47, wherein the applicator is a roller-ball applicator (a roll-on), a dispensing device for a cream or an ointment, a spray, a disposable or non-disposable applicator cloth, or a tissue or membrane.

49. A method for preventing or ceasing interaction of an allergen with a subject's skin region, the method comprising applying to the subject's skin region a formulation according to claim 41.

50. The method according to claim 49, wherein the formulation is applied by using a gel, an ointment, an emulsion, a thick cream, a liniment, a balsam, a lotion, a foam, a mask, a shampoo, tonic means, a cleaner, a spray, a hair spray, ear drops, eye drops, a conditioner, a roll-on, a powder including liquid powder, compact powder, cosmetic pencil, wet wipes or an application cloth comprising the formulation.

51. The method according to claim 49, wherein the formulation is applied by using an applicator comprising the formulation.

52. The method according to claim 49, for preventing or inhibiting a contact between an allergen and a skin region or binding of an allergen to an IgE epitope.

53. The method according to claim 49, wherein the formulation is applied onto a subject's skin region prior to exposure to an allergen or to a subject susceptible to having an allergic reaction.

54. The method according to claim 53, wherein the allergen is capable of inducing, promoting, or stimulating allergy.

55. The method according to claim 49, for preventing an allergen-mediated disease or disorder or form arresting deterioration of an existing condition due to continued exposure to the allergen.

56. The method according to claim 55, wherein the disease or disorder is caused by binding of IgE to an allergen causing immediate Type I allergic reaction.

57. The method according to claim 56, wherein the disease or disorder is selected amongst inflammatory, allergic and non-allergic diseases or disorders of the skin.

58. The method according to claim 57, wherein the disease or disorder is selected from hives (urticaria), eczema, angioedema, onchocercal dermatitis, dermatitis, atopic dermatitis, contact dermatitis and swelling.

59. A method for preventing atopic dermatitis in a subject, the method comprising applying to a skin region of the subject an amount of a formulation according to claim 41.

60. An applicator comprising a formulation according to claim 41.
